(19) United States
(12) Patent Application Publication
(10) Pub. No.: US 2010/0298907 A1
(43) Pub. Date: Nov. 25, 2010

(54) CORTICAL STIMULATOR METHOD AND APPARATUS

(75) Inventors: Daniel Lombardi, Verona, WI (US); Linda Quinlivan, Lewisville, NC (US); Bjorn Nelson, Verona, WI (US); Eric Garz, Lodi, WI (US); Ron Schluter, New Glarus, WI (US); Lucas Garsha, Verona, WI (US)

Correspondence Address: Carefusion C/O Wagner Blecher LLP, 123 Westridge Drive, Watsonville, CA, 95076, US

(73) Assignee: CareFusion Neurocare, San Diego, CA (US)

(21) Appl. No.: 12/766,270

(22) Filed: Apr. 23, 2010

Related U.S. Application Data
(60) Provisional application No. 61/172,372, filed on Apr. 24, 2009.

(51) Int. Cl. A61N 1/08; A61N 1/36

(52) U.S. Cl. 607/45

(57) ABSTRACT

A cortical stimulator system is provided. The system may include; a stimulation device having a switch configured to selectively control various electrodes; and a user interface device operatively connected to the stimulation device for controlling the electronic switch and stimulation device, the cortical stimulator system configured to provide a report of provided stimulation. A method of operating a cortical stimulator may be provided. The method may include: connecting a set of probes to the cortical stimulator, selecting parameters regarding a signal to be sent to the set of probes, sending a signal to the set of probes; observing the response of a subject having the set of probes contacting the subjects brain when the signal is sent to the probes, entering the observed response into the cortical stimulator, associating the response to a specific set of probes, and generating a report describing the response and associated probes.

### CROSS-REFERENCE TO RELATED APPLICATIONS

This application claims priority to provisional U.S. patent application entitled, Cortical Stimulator Method and Apparatus, filed Apr. 24, 2009, having Ser. No. 61/172,372, the disclosure of which is hereby incorporated by reference in its entirety.

### FIELD OF THE INVENTION

The present invention relates to cortical stimulators and the like.

### BACKGROUND OF THE INVENTION

Cortical stimulation has been performed as part of a pre-surgical work up for decades, and has been well documented and clinically accepted. Cortical stimulation is typically achieved by means of direct stimulation of the cortex with biphasic constant current pulses being delivered by means of a bipolar probe, typically during brain surgery of a patient, or through intracranial electrodes during long-term monitoring. Functional brain mapping identifies critical functional regions of the brain including the motor area, which controls movement; somatosensory area, which controls sensation; and expressive and receptive language areas, which control speech and comprehension. By mapping the brain, the neurosurgeon can find a balance between tumor or epileptogenic foci resection and potential damage to critical brain areas that would affect patient quality of life.

Stimulation through a grid electrode is typically awkward, as the electrodes must be switched from the amplifier to the stimulator and back, either through a switchboard or manually, which is labor intensive and extremely error prone. In addition, brain maps which display the results of the stimulation, in terms of ictal, inter-ictal and functional responses, are typically hand-drawn.

Accordingly, there is a need and desire for a cortical stimulator having electronic electrode switching, stimulation capability, software integration and/or report generation.

### SUMMARY OF THE INVENTION

Embodiments of the present invention advantageously provide a cortical stimulator having electronic electrode switching, stimulation capability, and software integration and/or report generation.

In accordance with some embodiments of the invention, a cortical stimulator system is provided. The system may include; a stimulation device having a switch configured to selectively control various electrodes; and a user interface device operatively connected to the stimulation device for controlling the electronic switch and stimulation device, the cortical stimulator system configured to provide a report of provided stimulation.

In accordance with some embodiments of the invention, a method operating a cortical stimulator may be provided. The method may include: connecting a set of probes to the cortical stimulator, selecting parameters regarding a signal to be sent to the set of probes, sending a signal to the set of probes; observing the response of a subject having the set of probes contacting the subjects brain when the signal is sent to the probes, entering the observed response into the cortical stimulator, associating the response to a specific set of probes, and generating a report describing the response and associated probes

Before explaining at least one embodiment of the present invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a perspective schematic view of a cortical stimulator in accordance with an embodiment of the present invention.

FIG. 2 is a perspective schematic view of a stimulus control unit in accordance with an embodiment of the present invention.

FIG. 3 is a bottom schematic view of the FIG. 2 stimulus control unit.

FIG. 4 is a block diagram of electronics associated with a stimulus control unit in accordance with an embodiment of the present invention.

FIG. 5 is a perspective schematic view of a portion of a cortical stimulator in accordance with an embodiment of the present invention.

FIG. 6 is a perspective bottom view of the portion of the cortical stimulator shown in FIG. 5.

FIG. 7 is a block diagram of a stimulus switching unit in accordance with an embodiment of the present invention.

FIG. 8 is a graph showing a biphasic waveform in accordance with an embodiment of the present invention.

FIG. 9 is a schematic diagram of one embodiment of cortical stimulator system in an OR probe biphasic mode.

FIG. 10 is a schematic diagram of one embodiment of cortical stimulator system in an electrode biphasic mode.

FIG. 11 is a schematic diagram of an embodiment of cortical stimulator system in an electrode biphasic mode.

FIG. 12 is a schematic diagram of an embodiment of cortical stimulator system in an electrode biphasic mode.

FIG. 13 is a schematic diagram of an embodiment of cortical stimulator system in a stand alone configuration.

FIG. 14 is a schematic diagram of an embodiment of cortical stimulator system including a computer.

FIG. 15 is a schematic diagram of an embodiment of cortical stimulator system including a laptop type computer.

FIG. 16 shows a table of error codes and the meaning of the error codes.

FIG. 17 is a perspective schematic view of a amplifier for a cortical stimulator system in accordance with an embodiment of the present invention and shows an enlargement of part of the amplifier.

FIG. 18. shows various settings for a channel selector for the amplifier shown in FIG. 17.

FIG. 19. is a table showing an LED light configuration indicating which LED lights are illuminated when which channels for the amplifier of FIG. 17 are active.

FIG. 20 is a flow chart illustrating steps in a method of operating a cortical stimulator.

### DETAILED DESCRIPTION

In the following detailed description, reference is made to the accompanying drawings, which form a part hereof and show by way of illustration specific embodiments in which the invention may be practiced. These embodiments are described in sufficient detail to enable those skilled in the art to practice them, and it is to be understood that other embodiments may be utilized, and that structural, logical, processing, and electrical changes may be made. The progression of processing steps described is an example; however, the sequence of steps is not limited to that set forth herein and may be changed as is known in the art, with the exception of steps necessarily occurring in a certain order.

Embodiments of a cortical stimulator of the present invention include a complete system of hardware and software integrated to provide comprehensive biphasic constant current stimulation with trains of stimulation pulses while monitoring patient electroencephalogram (EEG) for real-time electrophysiological responses. This complete system may be combined with the ability to electronically select any pair of, for example, up to 128 grid and/or strip electrodes. Stimulation initiation and other parameters can be controlled from either the hardware or software control panel.

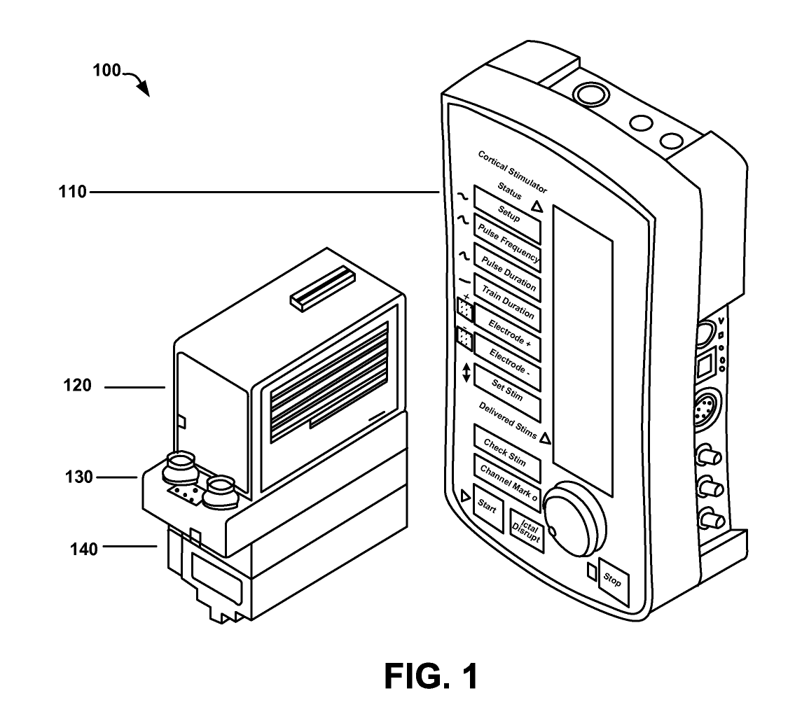

The invention will now be described with reference to the drawing figures, in which like reference numerals refer to like parts throughout. FIG. 1 is a perspective schematic view of a cortical stimulator in accordance with an embodiment of the present invention. A cortical stimulator 100 may include a stimulus control unit 110, a first amplifier 120, a stimulus switching unit (SSU) 130, and a second amplifier 140.

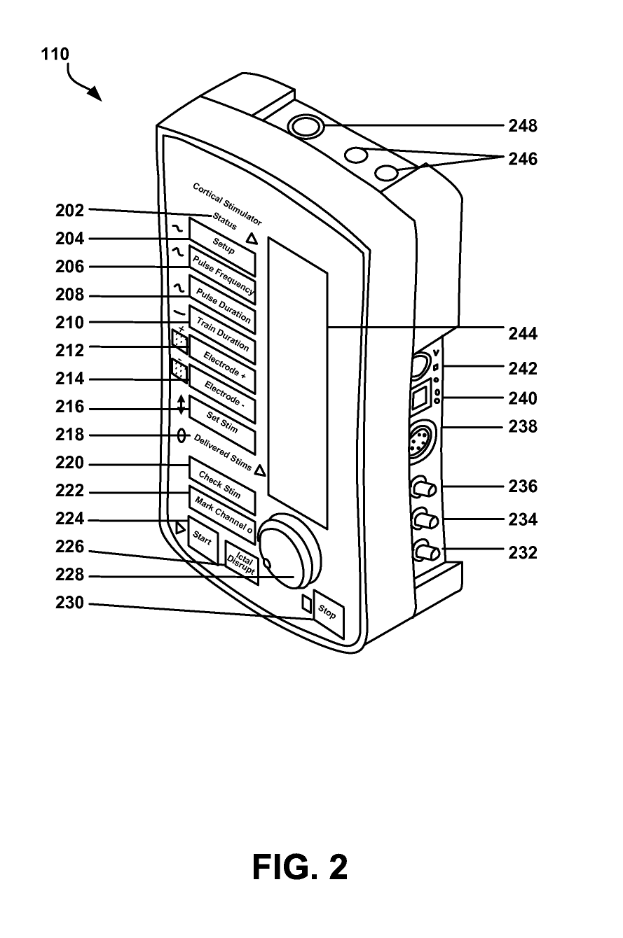

FIG. 2 is a perspective schematic view of a stimulus control unit in accordance with an embodiment of the present invention. The stimulus control unit (SCU) 110 may include a status indicator 202 for showing a current status of the stimulator 100. The various status conditions may include a set-up mode, a ready mode and a Stim-on (where stimulation may be actually occurring) mode. A setup selector 204 on the stimulus control unit 110 may be for allowing a user to change parameters of the stimulus control unit 110. For example, some changes to the SCU 110 may include changing between a probe biphasic and a electrode biphasic mode (these modes will be discussed later below). Selecting between a numeric and montage label sets, and a list of languages messages from the SCU 110 will appear. The SCU 110 may include a pulse frequency selector 206 for viewing and/or setting a rate at which pulses are delivered. A typical rate is 50 Hz but other rates may also be used.

The SCU 110 may include a pulse duration selector 208 for viewing and/or setting a length of time for each pulse. The actual pulse length may be twice the pulse duration. Example pulse durations may range from 100-1000 uSec, however other durations may be used. A train duration selector 210 may be used for viewing and/or setting a maximum stimulus duration. A train duration of 5 seconds is typical, however other durations my be used. A single train duration or an externally controlled trigger (such as by a computer connected to the SCU 110) may be selected.

The stimulus control unit 110 may further include electrode channel selectors 212, 214. The channel selectors 212, 214 may used to switch a probe or electrode from anode to cathode or vise versa. In some embodiments the channel selectors 212, 214 may select between 1-64 channels or 1-128 channels if a second SSU (explained further below) is connected to the SCU 110. Selecting a channel will select which electrodes will received the stimulus. Rotation of the selector knob 228 may select a channel once the channel selectors 212 and/or 214 are actuated. The SCU 110 is equipped with a set stimulus selector 216 for setting a current level to be applied to a patient. A base line up to about 8 mAmps or less is typical although other levels may be used. The selector knob 228 may be used to adjust the value of the current after the stimulus selector 216 is actuated. A delivered stimulus indicator 218 displays the stimulation level being delivered to a patient. An LED may illuminate to indicate when stimulation is being delivered. A stimulus check selector 220 can apply a selected stimulus to an internal load (not shown) to verify correct operation. In some optional embodiments LED lights may illuminate when this feature is enabled. The actual current that is delivered is displayed to a delivered stim display field.

The stimulus control unit 110 may further include a mark channel selector 222 for indicating which channel or channels are selected. The mark channel selector 222 may be depressed by a user when the channel is selected. The SCU 110 has a start selector 224 for delivery of the stimulation pulse (train, single or single). In one embodiment, the start selector 224 may function only when the cortical stimulator 100 is in a “ready state,” i.e., ready to provide cortical stimulation and the external trigger function is not being used. The SCU 110 may include an ictal disrupt selector 226. When actuated the ictal disrupt selector 226 may repeat a first pulse in a pulse train. The selector input 228 may allow a user to scroll through various options accessed by any of the various selectors and indicators, for example, the electrode channel selectors 212, 214 and set stimulus selector 216. A stop selector 230 may interrupt stimulation. Trigger in, trigger out, and synchronization connector inputs 232, 234, 236 may allow external control of the stimulus control unit 110. A serial port 238 may allow a serial connection for an external interface to other devices or a computer. A USB port 240 may allow a USB connection for an external interface, for example, for service diagnostics and optionally for a computer interface. A remote start/stop port 242 may allow remote control of starting and/or stopping the cortical stimulator 100.

The stimulus control unit 110 may further include a display 244 for displaying any information and/or parameters pertinent to operation to the user including any information generated by any of the above-described indicators and selectors. The display 244 may be, for example, a liquid crystal display (LCD).

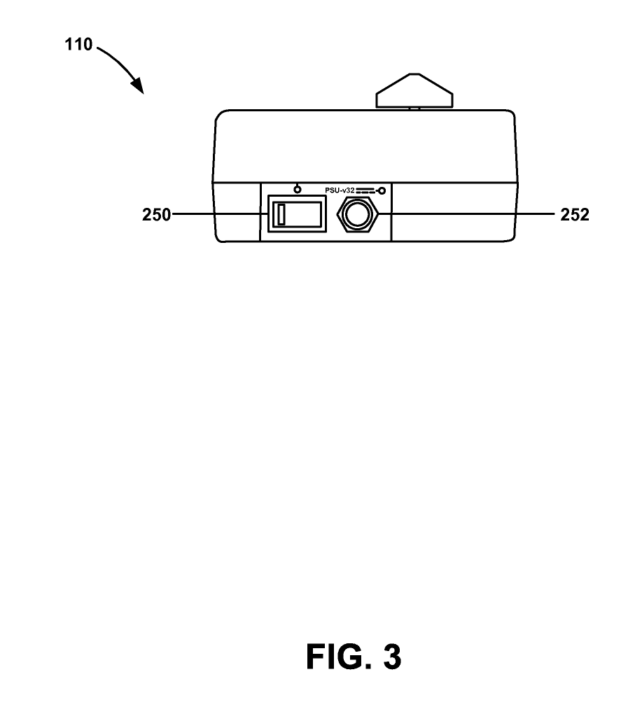

FIG. 3 is a bottom schematic view of the FIG. 2 stimulus control unit. The stimulus control unit 110 may further include a power switch 250 and a power supply connection 252. It should be appreciated that any of the FIGS. 2 and 3 elements may be located at any appropriate location. The illustrated elements are not limited to the locations, sizes, or geometries shown. For example, although the selector input 228 is shown as a knob, it may be a joystick, scroll wheel, arrow buttons, or any input device suitable for the desired function.

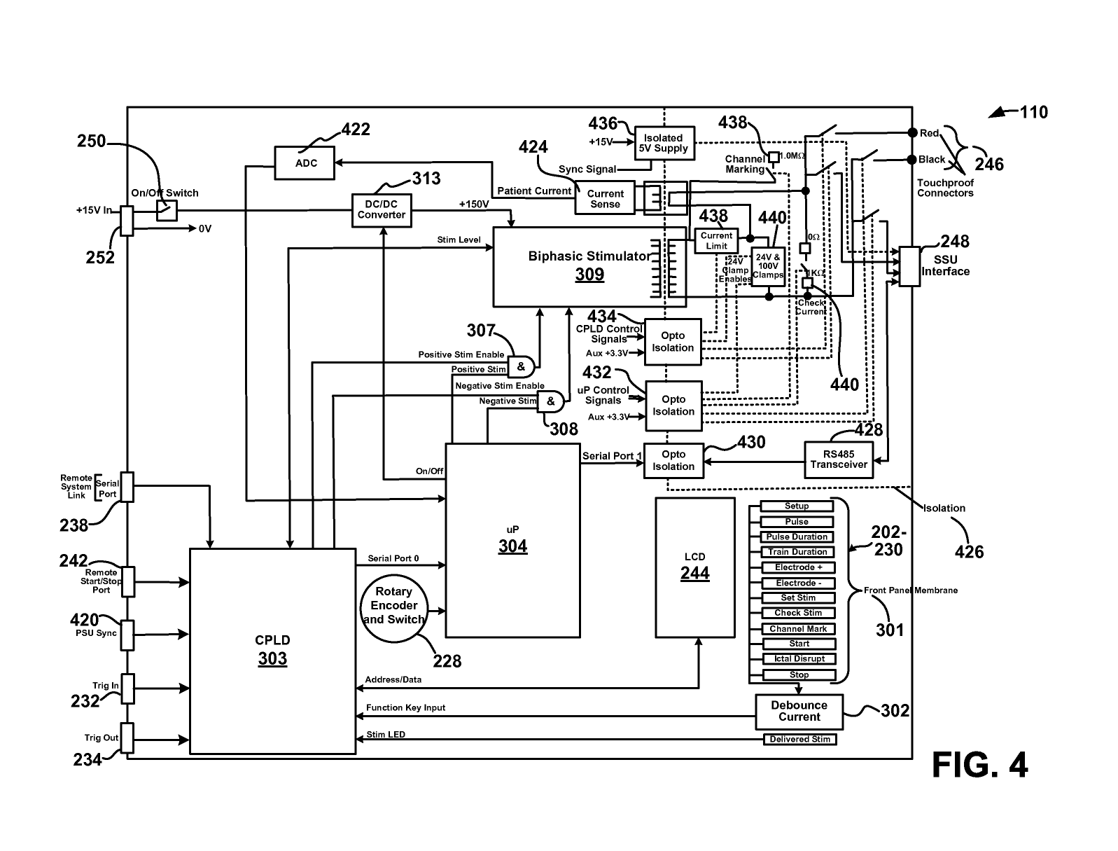

FIG. 4 is a block diagram of a stimulus control unit 110 in accordance with an embodiment of the present invention. The stimulus control unit 110 may include a front panel membrane 301 on which the selectors 202-230 may be located. It should be appreciated that the markings on the membrane may be graphical or text in any appropriate language. In one embodiment of the present invention, both text and graphics are provided such that a user who is not familiar with the language in which the text is written may understand and operate the cortical stimulator 100. Inputs made to the front panel membrane 301 may feed into at least one debounce circuit 302 for debouncing and stabilizing user inputs. A programmable logic device (PLD) 303, for example, a complex programmable logic device (CPLD) or a field-programmable gate array (FPGA), may receive inputs from the indicators and selectors described above via the debounce circuit 302 or directly from the front panel membrane 301.

Address, data, and control information may be passed between the PLD 303 and a processor (uP) 304 for controlling operations of the stimulus control unit 110. The processor 304 may also control the display 244. The selector input 228 may provide an input directly to the processor 304. The processor 304 may provide outputs to a positive stim AND logic 307 and to a negative stim AND logic 308, which provide a respective positive and negative input to a biphasic stimulator 309.

The PLD 303 may be electrically connected to the trigger in and trigger out connector inputs 232, 234.

When the power switch 250 is set to an “ON” position, power is provided via the power supply connection 252, which may be passed through other circuitry to a direct current to direct current (DC/DC) converter 313. The DC/DC converter converts a voltage level received, e.g., +15 V, into a voltage level required by the biphasic stimulator 309, e.g., +150 V. The biphasic stimulator 309 provides stimulation to the patient based on controls sent from the PLD 303 and processor 304.

As shown in FIG. 4, a PSU sync 420 may be attached to the CPLD 303. An ADC 422 is located between the Current sense 424 and the uP 304. Some embodiments in accordance with the invention may include an isolation circuit 426 to reduce the likelihood of current leaking from the SCU 110 to the subject. In some embodiments of the invention, the isolation circuit (sometimes referred to as a blocking circuit) may prevent current from leaking into amplifier inputs associated with electrodes configured to receive current. This blocking or isolation feature may result in more current available for electrodes intended to receive current and less amplifier recovery time.

The isolation circuit 426 may include a RS485 transceiver 428 connected to the SSU interface 248 and the Opto isolation 430. The Opto isolation 430 recieves an input from a serial port in the uP 304. An Opto isolation 432 may receive uP control signals and an Auxiliary +3.3V input. An Opto isolation 434 may receive CPLD control signals and an Auxiliary +3.3V input and may be connected to switches, a current limit 438 and 24V or 100V clamps 440, and a channel marking 438 as shown. An isolated 5V supply 436 may also be part of the isolation circuit 426 While example voltages have be described herein, it should be understood that these are examples only and other voltages may be used in accordance with the invention.

The microprocessor 304 used inside the SCU 110 may perform several tasks. For example, the microprocessor 304 may: enable a ±24V DC at output, set a Stim level, request positive stim pulses, request negative stim pulses, enable output relays to export the stimulating current, and monitor the stimulating current via in-built 16-bit ADC. The microprocessor may also monitor the state of the front panel switches 202-230, monitor the position of a rotary encoder and associated switch and the present information on the LCD 244. The microprocessor 304 may interact with a remote computer via the RS232 link. The microprocessor 304 may interact with a remote computer to validate parameter settings and return status information. The microprocessor 304 may interact with the SSU 130 to set the SSU 130 configuration and monitor the SSU 130 status. The microprocessor 304 may also monitor the stim level and the +12 V and −15V voltage rails.

The microprocessor will access the LCD 249 and CPLD (complex programmable logic device 303) components via its external memory interface.

Assuming that the microprocessor 304 is functional, it will be able to check the operational status.

Before stimulation is activated, the microprocessor 304 will check that the stim intensity level, set by the 16-bit DAC, is at the expected level.

The microprocessor 304 may monitor the stimulator output current, even if it is not meant to be stimulating. If the output current is not within a set percentage of the expected output current, then the microprocessor 304 will switch off the stimulator circuit and de-energize the photo-mos relay.

The microprocessor 304 may have a supply voltage monitor that may be used to halt the processor in the event that the 3.3V voltage rail goes outside of the expected range.

The microprocessor 304 is interrupted by a timer on a regular basis (every 10 uS). Towards the start and end of the interrupt service routine, the microprocessor 304 refreshes registers within the CPLD 303. If this process does not take place, then the CPLD 303 will be able to interrupt any current flow by switching off some of the photo-mos relays in the stimulator output stage. The two stim enable outputs from the CPLD 303 may also be switched off and this, in turn, will guard against any stimulator pulses that are generated by the microprocessor 304 from having any further effect.

Complex programmable logic device (CPLD) 303 also inside the Stimulus Control Unit 110 is used to interface several signals to be microprocessor 304 and to monitor its operation.

The CPLD 303 will disable stimulation by inhibiting the stimulator pulses generated by the microprocessor 304 and by de-energizing one of the stimulator output relays.

The CPLD 303 is provided with its own reference oscillator for timing purposes, making it independent of the microprocessor system clock.

The CPLD 303 also monitors the frequency and duration of any stimulation. The stimulator configuration is written to registers within the CPLD 303 and it is the contents of these registers that are used to present data on the LCD 244. This latter process ensures that any defects within the memory inside the microprocessor 304 will not be propagated through to the CPLD 303 without being noticed either by a user operating the unit in the Local mode or by a system that interrogates the Stimulus Control Unit 110 remotely.

When stimulation is in progress, the CPLD 303 will check that the microprocessor 304 is generating the expected pulse train. If the microprocessor 304 deviates from what is expected, then the CPLD 303 will switch off its two stim enable outputs and this in turn will guard against any stimulator pulses that are generated by the microprocessor 304 from having any further effect. The CPLD 303 will also switch off some of the photo-mos relays in the output stage.

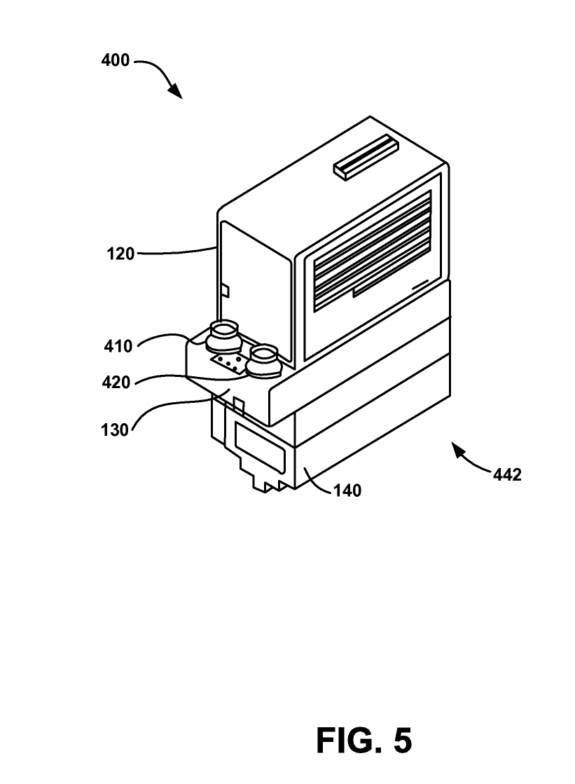

FIG. 5 is a perspective schematic view of a portion of a cortical stimulator in accordance with an embodiment of the present invention. A stimulus switching device 400 may include a first amplifier 120, a stimulus switching unit (SSU) 130, and a headbox 140. First and second cable connectors 410, 420 provide inputs to the stimulus switching device 400 via the stimulus switching unit 130.

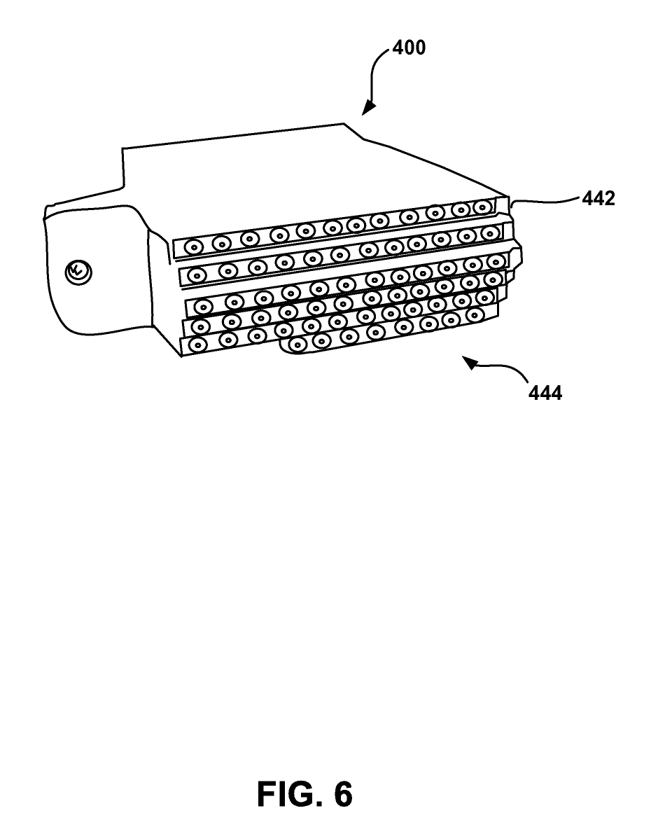

As shown in FIG. 6, at bottom 442 of the stimulus switching device 400 are multiple terminals 444. The multiple terminals 444 are configured to provide a place of various electrodes (not shown in FIG. 6) to plug in to. The electrodes may be part of a grid, matrix, or strips of electrodes. The electrodes may be inserted onto the brain of a subject undergoing a stimulation procedure.

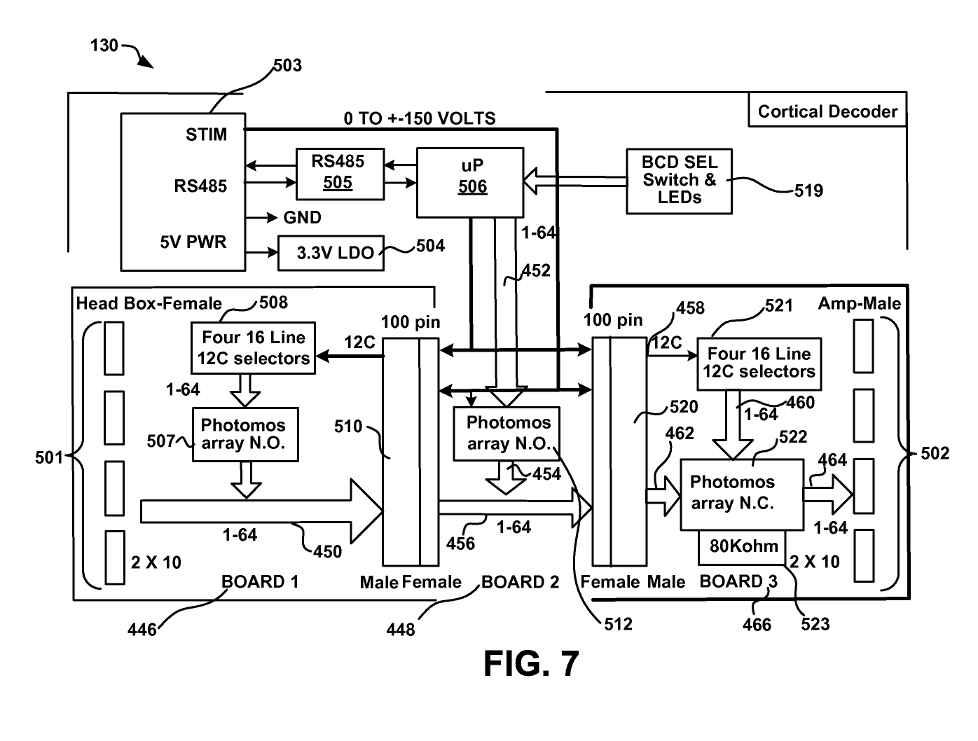

FIG. 7 is a block diagram of a stimulus switching unit 130 in accordance with an embodiment of the present invention. The stimulus switching unit 130 may include head inputs 501 from a head box 140. The output 502 of the stimulus switching unit 130 may be provided to an amplifier, e.g., the first amplifier 120, at a fourth output 464. In the illustrated example, there are between one (1) and sixty-four (64) input/output sets, i.e., channels.

A low-dropout (LDO) regulator 504 may receive an input from the stimulation input 503. The stimulation input 503 may communicate via a communications channel 505, e.g., an RS-485 communications channel, with a processor 506. The processor 506 can communicate with a first photoMOS array 507, i.e., an optical isolator that uses a short optical transmission path to transfer a signal between elements of a circuit, while keeping them electrically isolated. A transistor or other switching array may also be used. A BCD SEl. Switch and LEDs 519 are also operatively connected to the controller 506. A first input/output (I/O) expander 508 may be communicatively connected to the processor 506 for providing signals to the first photoMOS array 507. A reference voltage REF (for example 5V) may also be provided by the stimulation input 503 to the first photoMOS array 507. The above-described elements 503-508 may be provided on a first circuit board 446. Although some of the elements 503-508 may optionally be contained on second circuit board 448. An output from the photoMOS array 507 may be combined with the output from the head inputs 501 in a second output 450, which may be, for example, on a 100 pin board-to-board connector.

The second output 450 may be provided to a first male/female interface 510. The second photoMOS array 512 may also receive an outlet 452 from the processor 506. The second photoMOS array 512 may provide an output 454, which may be combined with the output 456 from the male/female interface 510 to the male/female interface 520.

The second male/female interface 520 may provide an output 458 to a third I/O expander 521, which may provide an output 460 to a third photoMOS array 522. The output 462 from the male/female interface 520 may be provided as a second input to the third photoMOS array 522, which may include a load 523, e.g., a resistor having a value of 80 kΩ. The third photoMOS array 522 may provide the fourth output 464, which may be, for example, on a 100 pin board-to-board connector. The third I/O expander 521 and the third photoMOS array 522 may be provided on a third circuit board 466.

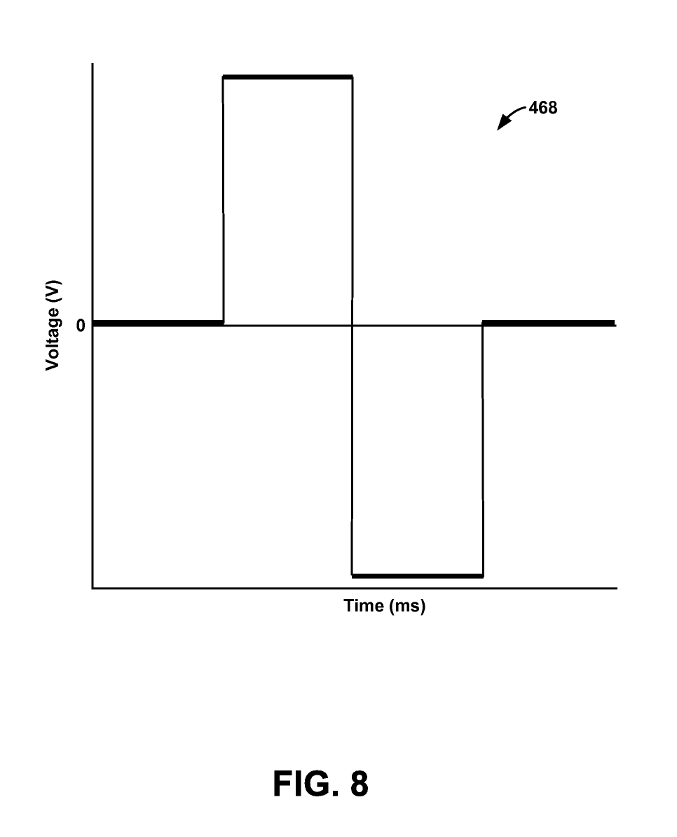

FIG. 8 depicts a graph showing a biphasic waveform 468 in accordance with an embodiment of the present invention. The biphasic waveform 468 is a pulse having positive and negative voltage for stimulating a patient over a period of a few milliseconds. It should be appreciated that the voltage levels, pulse time, and initial direction, i.e., positive or negative voltage, may be adjusted as required for the particular application within the scope of embodiments of the invention.

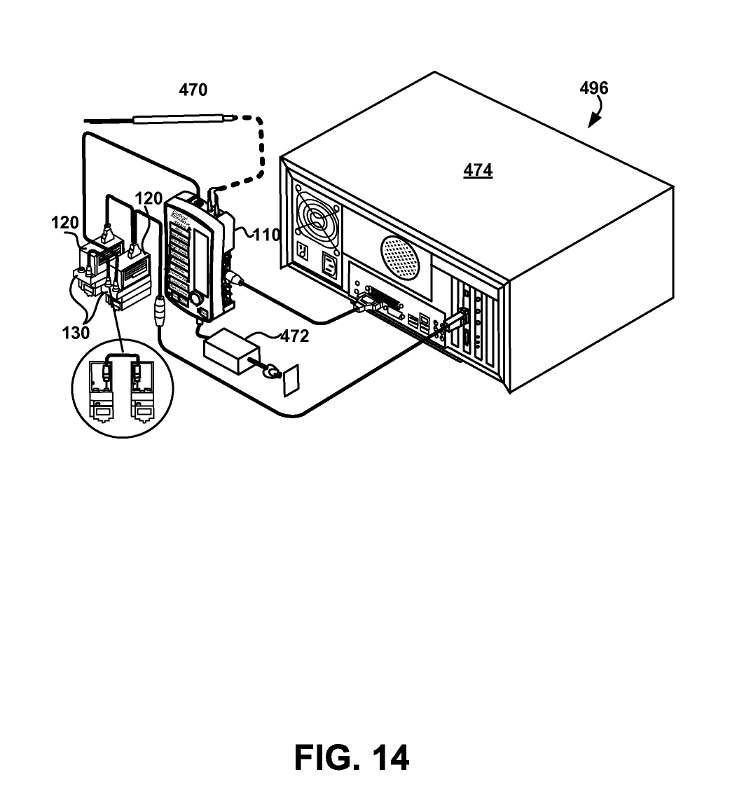

The cortical stimulator system describe herein may be used in at least two basic modes. A first mode may be referred to a an OR probe Biphasic mode and a second mode may be referred to as an Electrode Mode. As used herein, the terms “probe” and “electrode” are used interchangeably are not meant to be mutually exclusive. The OR probe Biphasic mode may be used when a set of probes (a cathode and an anode such as those 470 shown in FIG. 14) are moved from place to place on the brain of a subject during a procedure.

In the electrode mode, a series of probes (or electrodes) have been attached to the brain of a subject. The series of electrodes may be configured as pairs (an cathode and an anode) and arranged in a grid, matrix or in strips. The series of electrodes maybe be secured to the subject's brain so that they will remain in place as the subject moves about. In some instances the series of electrodes may have been placed earlier and may have been used in a procedure prior to the cortical stimulation procedure.

A software graphical user interface (GUI) may present the grid/strip electrode arrays shown on the brain view. The GUI facilitates ease of use. Pairs of electrodes can be selected for stimulation by pointing and clicking on the specific electrodes illustrated in the GUI. At the beginning of stimulation, the EEG acquisition window may open immediately, permitting the attending physicians an instant view of any seizure related, ictal and interictal activity (like “after discharges”, auras, and seizures) on all the electrodes including the pair being stimulated. Ictal/Interictal annotations can be made directly on the relevant electrodes as indicated by the observed EEG activity. In addition, a Functional Annotation field may be available to document any motor, sensory, speech and visual responses elicited by the stimulated pair of electrodes. The responses may be recorded as various colored bars linking the stimulated electrode pair combined with a legend that correlates to the specific function.

Electrodes stimulated and “cleared” may be marked with a gray border to avoid unintentional repetition of stimulation. Ictal and interictal responses may be indicated by filling in the corresponding electrode symbol with a specific color indicating the exact nature of the physiological response.

In addition to the features described above, other features may be included. Circuitry may be designed to block stimulation current from escaping into the amplifier, which assures that all of the current flows through the selected electrode pair and decreases amplifier recovery time post stimulation, which may be less than 1 second. A convenient small size may enable use as a hand held stand alone unit. The device may be used with a bipolar probe for manual brain mapping during surgery or with intracranial electrodes for bedside procedures. Two or more stimulus switching units may be coupled to electronically select additional electrodes and electrode pairs. In one embodiment of the present invention, two stimulus switching units are coupled to allow selection of up to 128 electrodes (i.e., 64 electrode pairs). It should be appreciated that additional or fewer units and electrodes may be used, as desired.

The device may also have a user-configurable pulse frequency, pulse duration, train duration, and current level, for example, respectively set by the pulse frequency selector 206, pulse duration selector 208, train duration selector 210, and set stimulus selector 216. Moreover, the stimulator may also include a “single stimulus pulse” mode allowing a single pulse to be generated, rather than a pulse train, e.g., selectable by the ictal disrupt selector 226. A “continuous stimulus pulse” mode may also be available for use, for example, with the bipolar probe. An actual reading of the current delivered may also be displayed, e.g., selectable by the stimulus check selector 220. A stimulus time remaining may count down to zero, or may count up, as appropriate, for example, to a preset time or without bound, which may be displayed, e.g., on the display 244. Continuous error detection may provide a high level of patient safety. There may also be an “active stimulation” indicator to indicate that stimulation is in progress, e.g., the status indicator 202. A “trigger out” may permit synchronization of additional equipment, e.g., the trigger out connector input 234.

The EEG acquisition amplifier and stimulus switching unit may be mechanically connected to form a single robust unit. When not needed for cortical stimulation, the unit can be used for routine long-term monitoring with no degradation of signal quality.

An ictal disrupt feature may stop after discharges before they can propagate into seizures which may result in premature termination of the session, which may be selectable by the ictal disrupt selector 226. Stimulus trains can be aborted prematurely with a “stop” button, e.g., the stop selector 230. A “check stim” feature may measure and verify accurate stimulator operation, e.g., selectable by the stimulus check selector 220. A “channel mark” feature may confirm that a correct electrode pair has been selected and stimulated, e.g., selectable by the mark channel selector 222. An annotation log may be automatically updated with stimulus settings. Multiple color coded functional and ictal event brain mapping with description legend.

A grid/strip editor may provide a complete list of available grid, strip, and depth electrodes to select from. Brain map size can be scaled to cover the range from infants to adults. Report results may be displayed by response category in a tabular format. An automatic report may provide visual documentation and an audit trail of stimulations and responses. Control of stimulus parameters may be available in multiple languages.

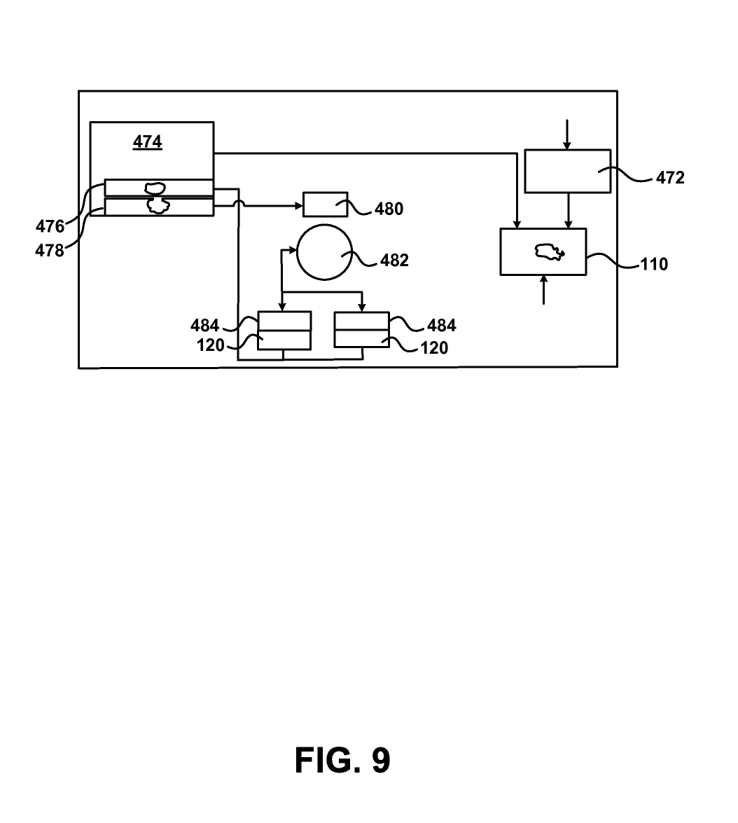

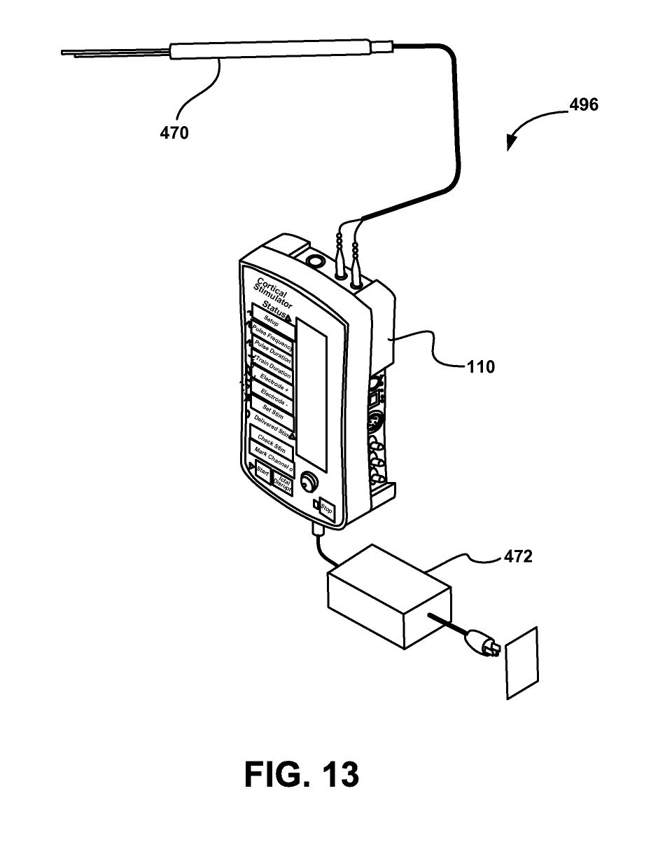

FIGS. 9-15 show various systems in accordance with different embodiments of the invention. FIGS. 9-12 and block diagrams and FIGS. 13-15 show the various componets in the system. FIG. 9 shows a hospital power supply 472 supplying power to the SCU 110. The SCU 110 is operatively connected to a computer 474 having an USB interface board 476 which permits the computer to communicate with a headbox 484 and the amplifier 120. The computer 474 has a digital video capability 478 that is operatively connected to a camera 480. The camera 480 may be used to record the procedure. Pictures from the camera 480 may by used in making a map of the brain to the subject.

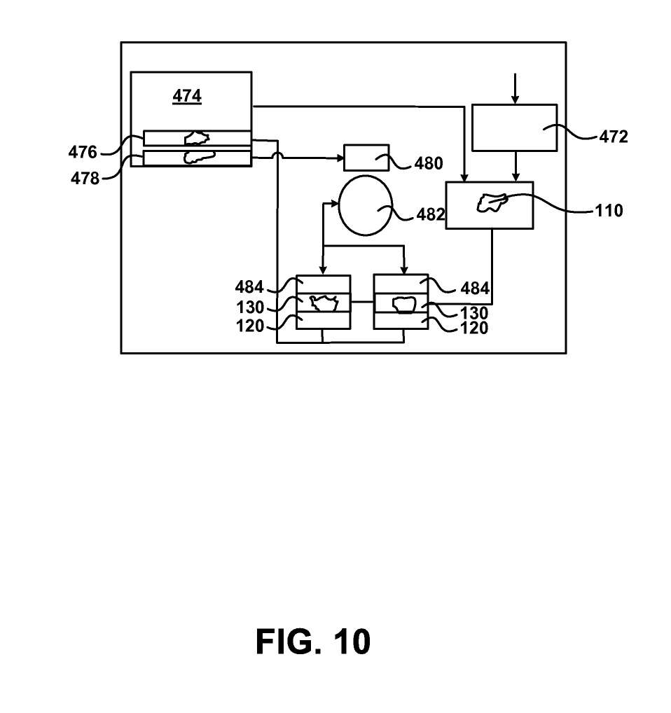

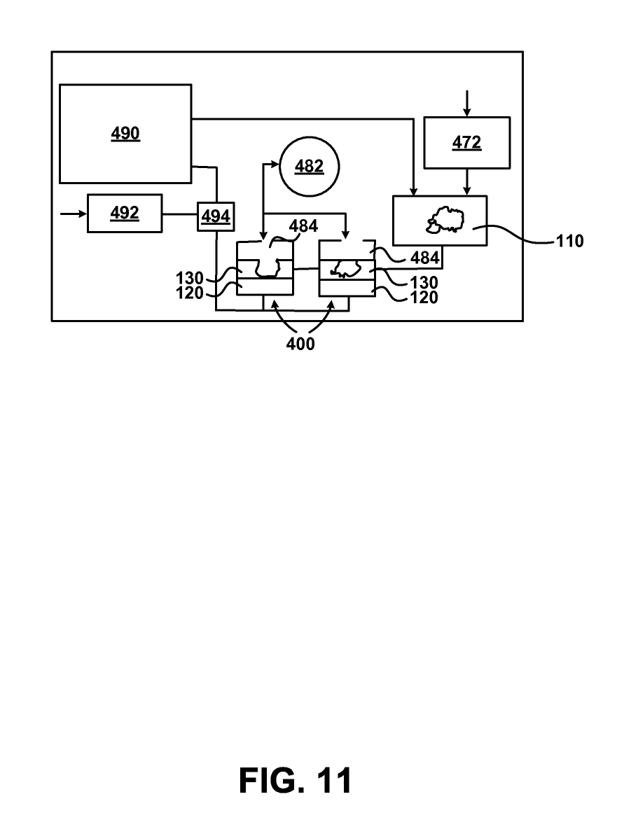

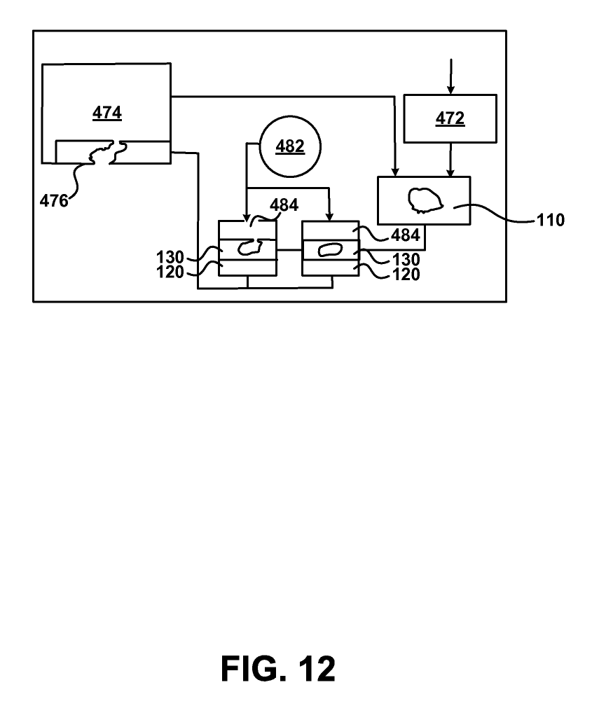

FIG. 10 is similar to FIG. 9 but adds the SSU 130 to provide the switching capability to the system. FIG. 11 is similar to FIG. 10 but uses a lap top computer 490 rather than a desktop type computer 474. To aid in communicating with the computer 490, an I box 494 with a power supply 492 are used. While the camera 480 is not shown it could be added to the components shown in FIG. 11. FIG. 12. is similar to that shown in FIG. 10 but does not show the digital video capability 478 and camera 480.

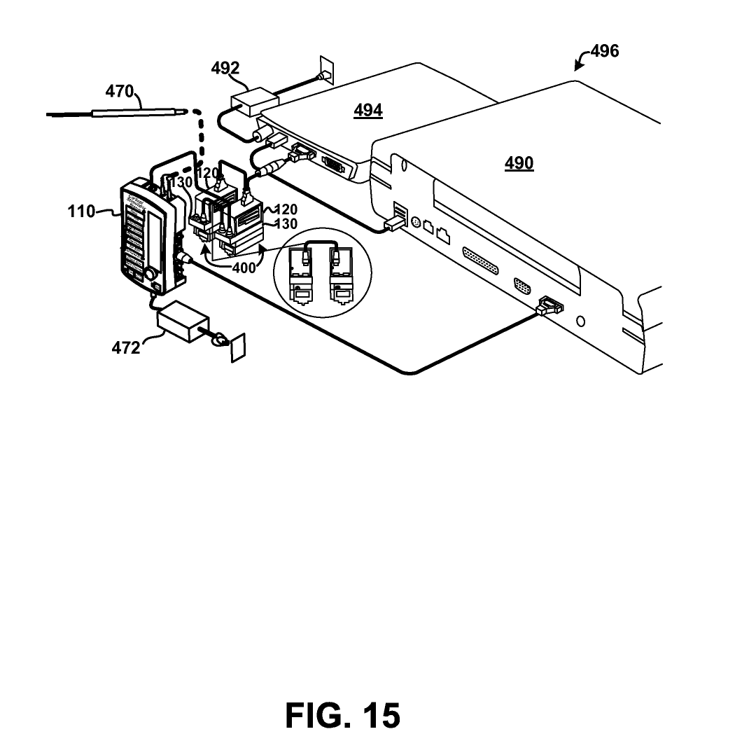

FIG. 13 shows a cortical stimulator system 496 used in a hand held manor. This system includes probes 470 connected to the SCU 110 which is connected to a power supply 472. The probes 470 may be 2.3 mm electrodes or equivalent. FIG. 14 shows a system 496 benefiting from the added capabilities of a computer 474. The probes 470 are connected to the SCU 110 which in turn is connected to a power supply 472 and a computer 474. The computer 474 and the SCU 110 are both operatively connected to the amplifiers 120 and SSUs 130. FIG. 15 is similar to FIG. 14 but uses a laptop type computer 490. The laptop computer 490 is connected to the stimulus switching device 400 via an I box 494. The I box 494 is connected to a power supply 492.

While FIGS. 14 and 15 show probes 470, it should be understood that a grid, matrix, or strips of electrodes 482 may be connected to the SSU 130 and used rather than probes 470.

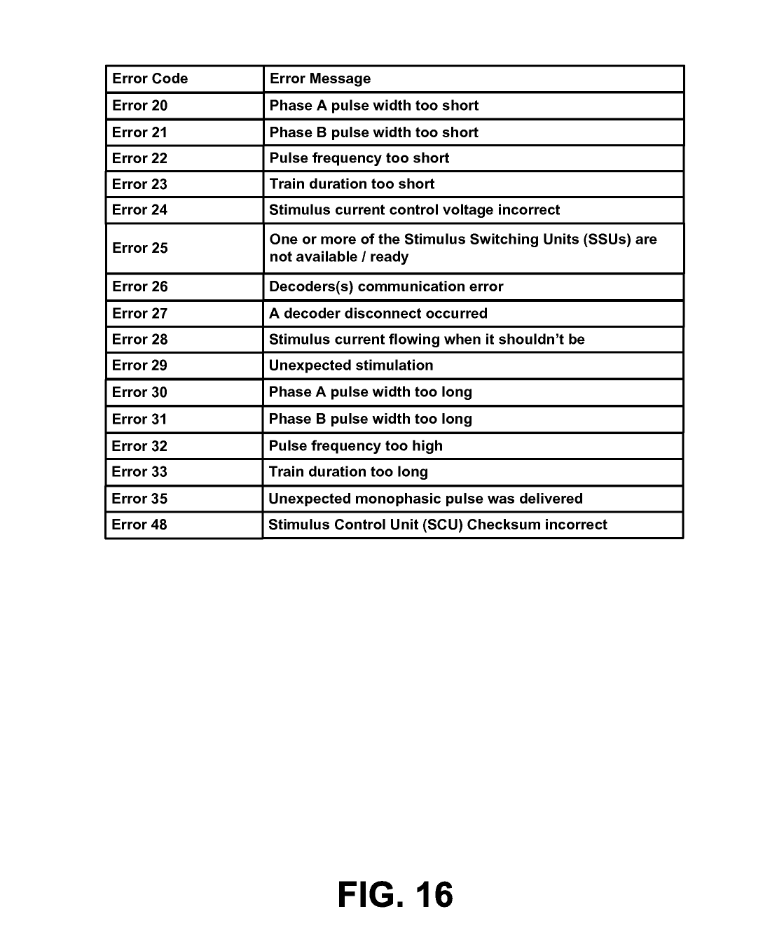

FIG. 16 shows a table of error codes and the corresponding meaning of the error codes. In the event that the SCU 110 or the system 496 detects an error or fault, the error code will be displayed to assist a user in troubleshooting.

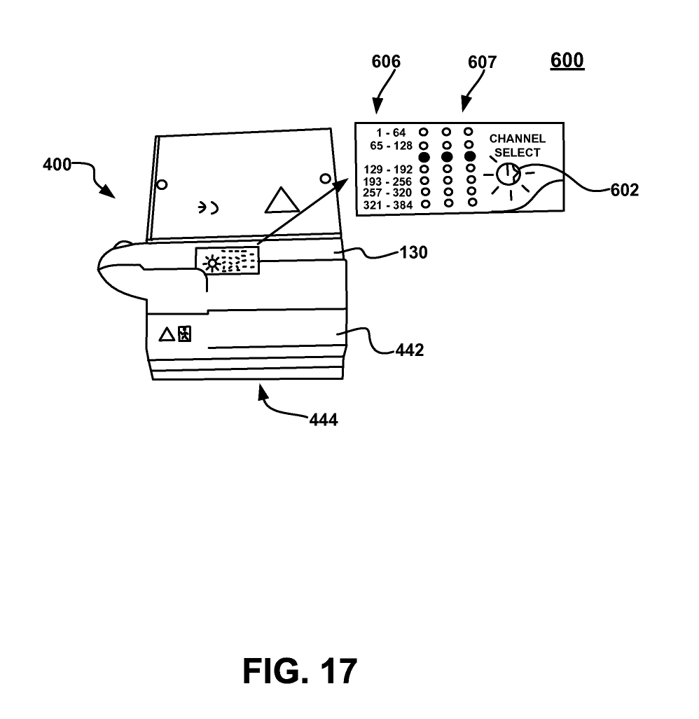

FIG. 17 shows stimulus switching device 400 having terminals 444 located at the bottom portion 442. A switch user interface 600 permits a user to switch which channels will be active. The channels may correspond to specific terminals 444. By manually setting the channel selector 602 a group of channels will be activated and may electronically be controlled by the SSU 130. LED lights 604 will illuminate and comparing the illuminated lights 604 with the indicators 606 a user will be able to tell which channels are active.

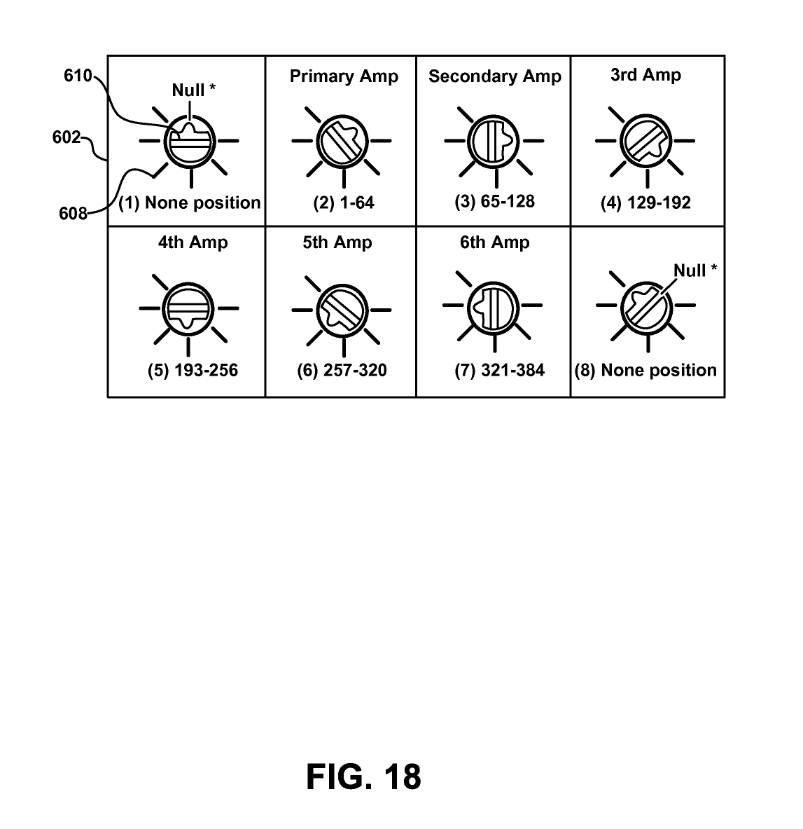

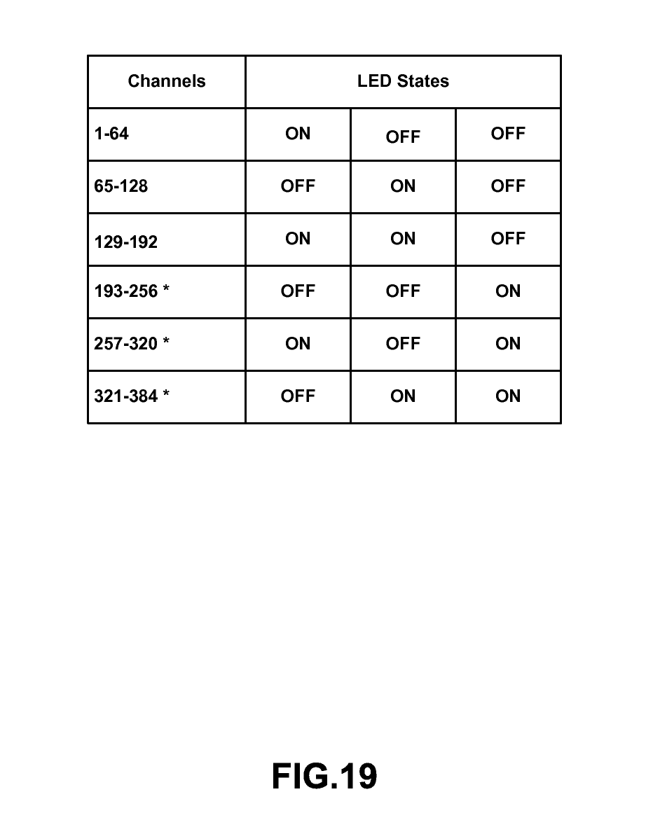

FIG. 18 show attitudes a channel indicator 602 may take. The rotator switch 610 will align with various indicator lines 608 to indicate what channels are selected. FIG. 19 is a table showing what LED lights 604 will be illuminated when specific channels are activated.

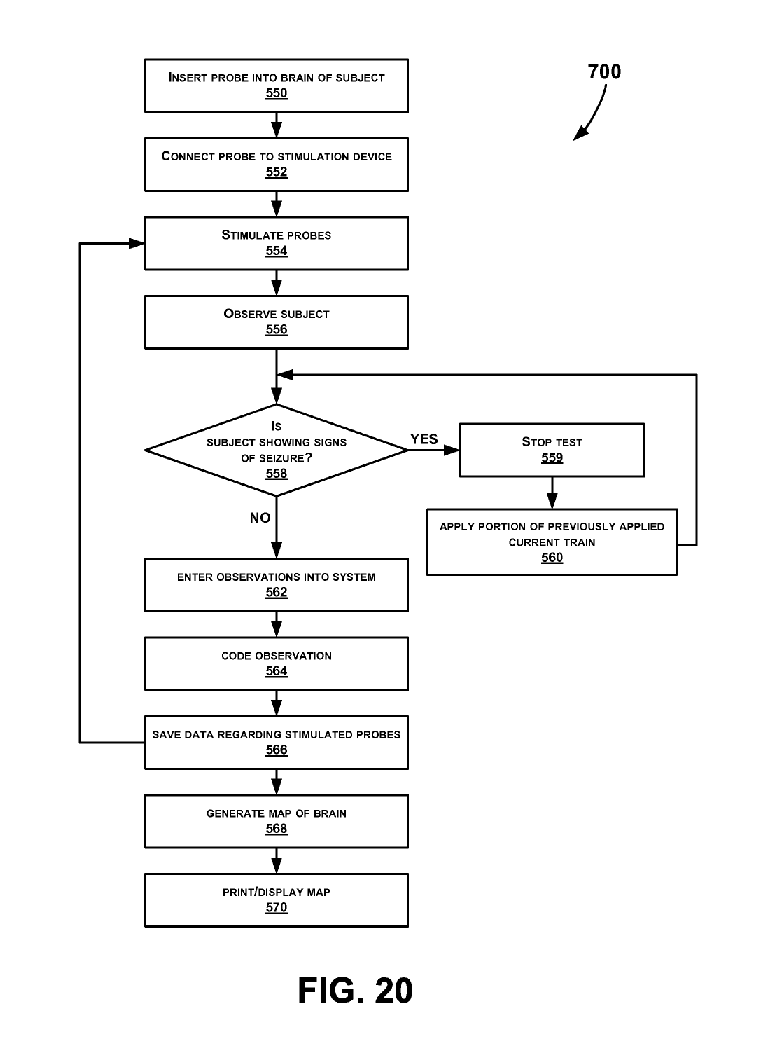

FIG. 20 shows a flow chart 700 showing various steps that may be accomplished while using the system 496. The steps shown in the flow chart 700 presuppose that the system 496 has been set up and the various parameters have been already set. The steps listed are not limited to the order they are shown and scribed. In step S50 the probes 470 (or a matrix/strip of electrodes 482 are inserted into the brain of a subject. In S52 the probes/electrodes 470/482 are connected to the stimulation device (optionally via a SSU). In S54 a selected pair of probes/electrodes 470/482 are stimulated by being sent a signal of current. In S56 the subject is observed. The subject may be asked to do a simple task and the subject's response will be observed. In S58 it is determined whether the subject is showing signs of an ictal response. If yes, the stimulation is stopped as shown in step S59. To prevent/abort or remediate the ictal response a portion of the previously applied current train may be applied to the probes/electrodes that precipitated the ictal response as shown in step S60.

If the ictal response has been aborted or none was observed, a user may enter the observations of the subject into the system as indicated in step S62. The user may associate a color with the observation. The system will associate the color and/or observation with the set of probes/electrodes and a portion of the brain that the probes/electrodes have been inserted. This information will be saved as shown in step S66. If additional probes/electrodes are to be stimulated, the method may then revert to S54 as shown. The information with be used to generate a map of the subject's brain as shown in step S68. As shown in step S70 the map may be printed or displayed. The map may be useful in assisting determining what parts of a subject's brain perform specific and or significant functions.

The processes and devices in the above description and drawings illustrate examples of some methods and devices of many that could be used and produced to achieve the objects, features, and advantages of embodiments described herein. Thus, they are not to be seen as limited by the foregoing description of the embodiments, but only limited by the appended claims. Any claim or feature may be combined with any other claim or feature within the scope of the invention. The many features and advantages of the invention are apparent from the detailed specification, and, thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and, accordingly, all suitable modifications and equivalents may be resorted to that fall within the scope of the invention.

What is claimed is:

1. A cortical stimulator system, comprising:
a stimulation device having a switch configured to selectively control various electrodes; and
a user interface device operatively connected to the stimulation device for controlling the electronic switch and stimulation device, the cortical stimulator system configured to provide a report of provided stimulation.

2. The cortical stimulator system of claim 1, further comprising an amplifier connected between the stimulation device and a matrix of probes.

3. The cortical stimulator system of claim 1, further comprising a computer operatively connected to the stimulation device, the computer having an input configured to permit a user to input observations regarding a subject and the system is configured to associate the input observations with a specific set of probes being stimulated and prepare a report regarding the specific set of probes and the input observations.

4. The cortical stimulator system of claim 3, wherein the computer is further configured to allow a user to associate a color with a set of probes and the report will display the associated colors.

5. The cortical stimulator system of claim 3, further comprising a display operatively connected to the computer, the display configured to display a representation of a subject's brain overlaid with data regarding the stimulation procedure.

6. The cortical stimulator system of claim 1, further comprising a display screen operatively connected to the stimulation device configured to display information regarding probes and operating parameters selected for the probes.

7. The cortical stimulator system of claim 1, wherein the user interface is configured to allow a user to adjust an aspect of a current sent to a set of probes, wherein the aspect include at least on of the following aspects: a level of current, a pulse frequency, a pulse duration, a train duration, and which probe is a cathode and which probe is an anode.

8. The cortical stimulator system of claim 1, further comprising a stop actuator operatively connected to the stimulation device and configured immediately stop sending stimulating signals to probes used in a stimulation procedure when the stop actuator is actuated.

9. The cortical stimulator system of claim 1, further comprising an ictal disrupt actuator operatively connected to the stimulation device and configured to cause the stimulation device to send an ictal disrupt signal to probes used in a stimulation procedure when the ictial disrupt actuator is actuated.

10. The cortical stimulator system of claim 9, wherein the ictal disrupt signal is at least part of the same signal that was applied to the probes immediately proceeding a stop actuator being actuated.

11. The cortical stimulator system of claim 9, wherein the system further includes multiple terminals to simultaneously plug into multiple sets of probes and the system further includes a switching device operatively connected to the multiple terminals, the switching device configured to selectively send signals to a selected set of probes.

12. The cortical stimulator system of claim 11, wherein the multiple sets of probes are connected to the switching device in parallel such that the switching device is configured to selectively send a signal to an individual set of probes while the multiple sets of probes are connected to the switching device.

13. The cortical stimulator system of claim 1, wherein the system is configured to display an error code to a user if the system detects an error within the operation of the system.

14. The cortical stimulator system of claim 1, further comprising a blocking circuit configured to prevent current from flowing to a set of probes connected to the system when the system is not set to send current to that set of probes.

15. A method operating a cortical stimulator comprising:
connecting a set of probes to the cortical stimulator;
selecting parameters regarding a signal to be sent to the set of probes;
sending a signal to the set of probes;
observing the response of a subject having the set of probes contacting the subjects brain when the signal is sent to the probes;
entering the observed response into the cortical stimulator;
associating the response to a specific set of probes; and
generating a report describing the response and associated probes.

16. The method of claim 15, further comprising stopping current from flowing to a set of probes attached to a subject if an ictal response is observed.

17. The method of claim 16, resending the signal sent to a set of probes immediately before the ictal response was observed.

18. The method of claim 15, further comprising electronically switching which set of probes is sent a signal

19. The method of claim 15, associating a color with an observed response and the set of probes that were stimulated resulting the response and inputting the response, and the associated color into the cortical stimulator.

20. The method of claim 15, electronically saving: the selected parameters regarding the signal sent to a specific set of probes; the which specific set of probes, and the entered observed response.

21. A cortical stimulator system, comprising:
a stimulation device configured to control at least one set of electrodes; and
an ictal disrupt actuator operatively connected to the stimulation device and configured to cause the stimulation device to send an ictal disrupt signal to the electrodes used in a stimulation procedure when the ictial disrupt actuator is actuated.

22. A cortical stimulator system, comprising:
a stimulation device configured to selectively send current to at least one set of probes; and
a blocking circuit configured to prevent current from flowing into inputs to an amplifier associated with probes selected to be stimulated.
